(19) United States
(12) Patent Application Publication
(10) Pub. No.: US 2011/0305715 A1
(43) Pub. Date: Dec. 15, 2011

(54) ANTIBODIES TO 3-HYDROXYCOTININE AND METHODS OF USE THEREOF

(76) Inventors: Lynn Y. Miao, Athens, OH (US); Jennifer G. Johnson, Athens, OH (US); Adriana Navarro, Jackson, OH (US); Paul Olivo, St. Louis, MO (US); David Scholl, Athens, OH (US)

(21) Appl. No.: 13/157,886

(22) Filed: Jun. 10, 2011

Related U.S. Application Data
(60) Provisional application No. 61/353,766, filed on Jun. 11, 2010.

(51) Int. Cl. A61K 39/395; A61P 25/34; G01N 33/98; C07K 16/44; C12N 5/16; C12N 5/0781

(52) U.S. Cl. 424/175.1; 435/345; 436/501; 435/7.9; 530/387.3; 530/389.8; 530/391.3; 435/7.92; 530/388.9

(57) ABSTRACT

The invention provides antibodies, and antigen-binding fragments thereof, that specifically bind to 3-hydroxycotinine (3HC). The invention also provides compositions containing these antibodies and fragments thereof, and to their use in diagnostic and therapeutic applications for diseases involving nicotine activity (e.g., smoking and/or smokeless tobacco use).

This application claims priority to co-pending U.S. provisional Application Ser. No. 61/353,766, filed Jun. 11, 2010, which is herein incorporated by reference in its entirety for all purposes.

### FIELD OF THE INVENTION

The invention relates to antibodies, and antigen-binding fragments thereof, that specifically bind to 3-hydroxycotinine (3HC). The invention also relates to compositions containing these antibodies and fragments thereof, and to their use in diagnostic and therapeutic applications for diseases involving nicotine activity (e.g., smoking and/or smokeless tobacco use).

### BACKGROUND OF THE INVENTION

The health risks of smoking have led to broad smoking cessation efforts in the workplace and other public settings. Current methods for measuring nicotine metabolism rely on gas chromatography (GC) and mass spectrometry (MS). However, GC/MS analysis is time-consuming, complex, expensive, and requires highly trained personnel. Thus, there remains a need for compositions and methods for rapid, inexpensive, easy to use, and specific tests for the measurement of nicotine metabolism, and for prediction of success of nicotine replacement products.

### SUMMARY OF THE INVENTION

The invention solves the need in the art by providing, in one embodiment, an isolated antibody, or an antigen-binding fragment thereof, that specifically binds to 3-hydroxycotinine (3-HC). While not intending to limit the type of antibody or antigen-binding fragment thereof, in one embodiment, the antibody is selected from the group of monoclonal antibody, chimeric antibody, recombinant antibody, humanized antibody, and an antibody displayed upon the surface of a phage. In an alternative embodiment, the antibody is a monoclonal antibody produced by a hybridoma cell. In a further alternative embodiment, the antibody, or the antigen-binding fragment thereof, does not substantially bind to a molecule selected from the group consisting of nicotine and nicotine metabolite. In another embodiment, the antigen-binding fragment is selected from the group consisting of a Fab fragment, a F(ab′)2 fragment, and a Fv fragment. In yet a further embodiment, the anti-3-hydroxycotinine antibody, or the antigen-binding fragment thereof, comprises a label.

The invention also provides an isolated monoclonal antibody, or an antigen-binding fragment thereof, produced by a hybridoma cell, wherein the antibody specifically binds to 3-hydroxycotinine (3-HC).

Also provided by the invention is a cell producing any one or more of the antibodies described herein, and/or any one or more of the antigen-binding fragments of the antibodies described herein. In one embodiment, the cell is a hybridoma cell. In a particular embodiment, the cell is a B-lymphocyte.

The invention also provides a kit comprising any one or more of the antibodies described herein, and/or any one or more of the antigen-binding fragments of the antibodies described herein. In one embodiment, the kit further comprises an anti-cotinine antibody, and/or an antigen-binding fragment thereof, that specifically binds to cotinine.

The invention additionally provides a method for detecting 3-hydroxycotinine in a sample, comprising a) providing i) any one or more of the antibodies described herein, and/or any one or more of the antigen-binding fragments of the antibodies described herein, and ii) a test sample, b) contacting the sample with the anti-3-hydroxycotinine antibody, or with the antigen-binding fragment thereof, and c) detecting binding of the anti-3-hydroxycotinine antibody, or binding of the antigen-binding fragment thereof, to the sample. In one embodiment, the method further comprises d) determining the level of 3-hydroxycotinine that binds to the anti-3-hydroxycotinine antibody, or binds to the antigen-binding fragment thereof. In another embodiment, the anti-3-hydroxycotinine antibody, and/or the antigen-binding fragment thereof, comprises a label. In one alternative embodiment, the step of detecting comprises an enzyme immunoassay. In an alternative embodiment, the method further comprises e) providing an anti-cotinine antibody, or an antigen-binding fragment thereof, that specifically binds to cotinine, f) contacting the sample with the anti-cotinine antibody, or with the antigen-binding fragment thereof, and g) determining the level of cotinine that binds to the anti-cotinine antibody or binds to the antigen-binding fragment thereof. In another embodiment, the method further comprises h) determining the ratio of the level of the 3-hydroxycotinine from step d) and the level of the cotinine from step g). In an additional embodiment, the method further comprises i) treating the subject to reduce one or more symptoms of nicotine dependency. In some embodiments, the treating step is selected from the group of administering to the subject one or more of (a) nicotine (e.g., chewable nicotine gum, nicotine patch, etc.), (b) anti-cotinine antibody, and (c) anti-hydroxycotinine antibody. In a particular embodiment, the anti-3-hydroxycotinine antibody is attached to a solid substrate. In a further embodiment, the detecting step comprises contacting the sample with a 3-hydroxycotine that comprises a label. In an alternative embodiment, the anti-3-hydroxycotinine antibody is in aqueous solution. In a further alternative embodiment, the detecting step comprises contacting the sample with a second antibody that specifically binds to the anti-3-hydroxycotinine antibody, or specifically binds to the antigen-binding fragment thereof, wherein the second antibody comprises a label. In yet another embodiment, at least one of the anti-3-hydroxycotinine antibody, the antigen-binding fragment of the anti-3-hydroxycotinine antibody, the anti-cotinine antibody, and the antigen-binding fragment of the anti-cotinine antibody, comprises a label, and the detecting step comprises detecting the label.

Also provided herein is a method for treating a disease associated with nicotine activity comprising administering to a subject in need thereof a therapeutically effective amount of any one or more of the antibodies described herein, and/or any one or more of the antigen-binding fragments of the antibodies described herein. In one embodiment, the method further comprises detecting a reduction in the level of 3-hydroxycotinine in a sample from the subject after the administering step compared to prior to the administering step. In another embodiment, the method further comprises detecting a reduction in one or more symptom of the disease following the administering step compared to prior to the administering step.

### BRIEF DESCRIPTION OF THE DRAWINGS

FIG. 1. Competitive microplate EIA for quantification of COT (A) and 3HC (B) by test principle #1. Different concentrations of COT or 3HC and fifteen other NIC metabolites were incubated with an immobilized COT MAb or 3HC MAb in the presence of a constant amount of COT-HRP or 3HC-HRP. The binding of the COT-HRP or 3HC-HRP to its corresponding MAb was detected by the reaction of the HRP with peroxidase substrate. The graph illustrates an inhibition curve that was formed with various concentrations of authentic COT (A) and 3HC (B). Apparent COT or 3HC concentrations are inversely related to the OD450 value. IC50 value for COT and 3HC is 66.1 ng/ml and 39.8 ng/ml respectively.

FIG. 2. Chemical structure of nicotine and its exemplary metabolites cotinine (COT), 3-hydroxycotinine (3HC), 5-hydroxycotinine (5HC), cotinine-glucuronide (COT-glucuronide), 3-hydroxycotinine-glucuronide (3HC-glucuronide), cotinine-N-oxide (COT-N-oxide) (CNO), Norcotinine, Nornicotine, Nicotine-N-oxide (NNO), and Nicotine-glucuronide. Also shown are tobacco alkaloids Anabaseine, Anatabine, Myosmine, Nicotelline, and Nicotyrine.

### DEFINITIONS

To facilitate understanding of the invention, a number of terms are defined below.

The terms “purified,” “isolated,” and grammatical equivalents thereof as used herein, refer to the reduction in the amount of at least one undesirable component (such as cell, protein, nucleic acid sequence, carbohydrate, etc.) from a sample, including a reduction by any numerical percentage of from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100%. Thus purification results in an “enrichment,” i.e., an increase in the amount of a desirable component cell, protein, nucleic acid sequence, carbohydrate, etc.) relative to the undesirable component.

The term “antibody” refers to an immunoglobulin (e.g., IgG, IgM, IgA, IgE, IgD, etc.). The basic functional unit of each antibody is an immunoglobulin (Ig) mononer (containing only one immunoglobulin (“Ig”) unit). Included within this definition are polyclonal antibody, monoclonal antibody, and chimeric antibody.

The variable part of an antibody is its “V domain” (also referred to as “variable region”), and the constant part is its “C domain” (also referred to as “constant region”) such as the kappa, lambda, alpha, gamma, delta, epsilon and mu constant regions. The “variable domain” is also referred to as the “F_{V} region” and is the most important region for binding to antigens. More specifically, variable loops, three each on the light (V_{L}) and heavy (V_{H}) chains are responsible for binding to the antigen. These loops are referred to as the “complementarity determining regions” (“CDRs” and “idiotypes.”

The immunoglobulin (Ig) monomer of an antibody is a “Y”-shaped molecule that contains four polypeptide chains: two light chains and two heavy chains, joined by disulfide bridges.

Light chains are classified as either (λ) or kappa (κ). A light chain has two successive domains: one constant domain (“C_{L}”) and one variable domain (“V_{L}”). The variable domain, V_{L}, is different in each type of antibody and is the active portion of the molecule that binds with the specific antigen. The approximate length of a light chain is 211 to 217 amino acids.

Each heavy chain has two regions, the constant region and the variable region. The There are five types of mammalian Ig heavy denoted a α, δ, ε, γ, and μ. The type of heavy chain present defines the class of antibody; these chains are found in IgA, IgD, IgE, IgG, and IgM antibodies, respectively. Distinct heavy chains differ in size and composition; α and γ contain approximately 450 amino acids, while μ and ε have approximately 550 amino acids. Each heavy chain has two regions, the constant region (“C_{H}”) and the variable (“V_{H}”) region. The constant region (C_{H}) is identical in all antibodies of the same isotype, but differs in antibodies of different isotypes. Heavy chains γ, α and δ have a constant region composed of three tandem (in a line) Ig domains, and a hinge region for added flexibility. Heavy chains μ and ε have a constant region composed of four immunoglobulin domains. The variable region (V_{H}) of the heavy chain differs in antibodies produced by different B cells, but is the same for all antibodies produced by a single B cell or B cell clone. The variable region of each heavy chain is approximately 110 amino acids long.

The term “specifically binds” and “specific binding” when made in reference to the binding of two molecules (e.g. antibody to an antigen, etc.) refer to an interaction of the two molecules that is dependent upon the presence of a particular structure on one or both of the molecules. For example, if an antibody is specific for epitope “A” on the molecule, then the presence of a protein containing epitope A (or free, unlabelled A) in a reaction containing labeled “A” and the antibody will reduce the amount of labeled A bound to the antibody. Thus, in one embodiment, the terms “specifically binds” and “specific binding” are relative term referring to the ratio (e.g., percentage) of the level of binding of an antibody to a first molecule (e.g., 3-hydroxycotinine) relative to the level of binding of the antibody to a second molecule (e.g., nicotine and/or nicotine metabolite and/or tobacco alkaloid). Thus, as used herein, “specific binding” of an antibody to a first molecule (e.g., 3-hydroxycotinine) refers to a level of binding of the antibody to a second molecule (e.g., nicotine and/or nicotine metabolite and/or tobacco alkaloid) that is 20% or less (including from 0% to 20%, from 0% to 19%, from 0% to 18%, from 0% to 17%, from 0% to 16%, from 0% to 15%, from 0% to 14%, from 0% to 13%, from 0% to 12%, from 0% to 11%, from 0% to 10%, from 0% to 9%, from 0% to 8%, from 0% to 7%, from 0% to 6%, from 0% to 5%, from 0% to 4%, from 0% to 3%, from 0% to 2%, and from 0% to 1% less) than the level of binding of the antibody to the first molecule. Said differently, “specific binding” of an antibody to a first molecule (e.g., 3-hydroxycotinine) refers to a level of binding of the antibody to the first molecule that is at least five (5) fold greater (including from 5 to 1,000, from 5 to 500, from 5 to 100, from 5 to 50, from 5 to 25, and from 5 to 10 fold greater) than the level of binding of the antibody to a second molecule (e.g., nicotine and/or nicotine metabolite and/or tobacco alkaloid). In one embodiment, the level of binding of an antibody to a molecule is determined using the IC50 value. For example, the exemplary anti-3-hydroxycotinine antibody 22H9C7 specifically binds to 3-hydroxycotinine since the IC50 (>5,000 ng/mL) of binding of the antibody to nicotine, cotinine (COT), 5-hydroxycotinine (5HC), cotinine-glucuronide (COT-glucuronide), 3-hydroxycotinine-glucuronide (3HC-glucuronide), cotinine-N-oxide (COT-N-oxide) (CNO), Norcotinine, Nornicotine, Nicotine-N-oxide (NNO), Nicotine-glucuronide, Anabaseine, Anatabine, Myosmine, Nicotelline, and Nicotyrine is less than 1% of the IC50 (39.8 ng/mL) of binding of the antibody to 3-hydroxycotinine (Table 3). The term “capable of binding” when made in reference to the interaction between a first molecule (such as antibody, polypeptide, glycoprotein, nucleic acid sequence, etc.) and a second molecule (such as antigen, polypeptide, glycoprotein, nucleic acid sequence, etc.) means that the first molecule binds to the second molecule in the presence of suitable concentration of salts, and suitable temperature, and pH. The conditions for binding molecules may be determined using routine and/or commercially available methods.

The term “does not substantially bind” when in reference to the interaction between a first molecule (e.g., antibody) and a second molecule (e.g., nicotine metabolite and/or tobacco alkaloid) means that the first molecule does not specifically bind to the second molecule, in that the interaction (if any) between the first and second molecules, is not dependent upon the presence of a particular structure on one or both of the molecules. Thus, in one embodiment, the term “does not substantially bind” is a relative term referring to the ratio (e.g., percentage) of the level of binding of an antibody to a first molecule (e.g., nicotine and/or nicotine metabolite and/or tobacco alkaloid) relative to the level of binding of the antibody to a second molecule (e.g., 3-hydroxycotinine). Thus, as used herein, an antibody that “does not substantially bind” to a first molecule (e.g., nicotine and/or nicotine metabolite and/or tobacco alkaloid) refers to a level of binding of the antibody to the first molecule that is more than 20% greater (including from 21% to 100,000%, 21% to 10,000%, 21% to 1,000%, 21% to 500%, 21% to 100%, 21% to 50%, greater) than the level of binding of the antibody to a second molecule (e.g., 3-hydroxycotinine). Said differently, an antibody that “does not substantially bind” to a first molecule (e.g., nicotine and/or nicotine metabolite and/or tobacco alkaloid) refers to a level of binding of the antibody to the first molecule that is less than five (5) fold greater (including from 0 to 4.5, from 0 to 4.0, from 0 to 3.5, from 0 to 3.0, from 0 to 2.5, from 0 to 2.0, from 0 to 1.5, from 0 to 1.0, and from 0 to 0.5 fold greater) than the level of binding of the antibody to a second molecule (e.g., 3-hydroxycotinine). In one embodiment, the level of binding of an antibody to a molecule is the determined using the IC50 value. For example, the exemplary anti-3-hydroxycotinine antibody 22H9C7 does not substantially bind to any one of nicotine, cotinine (COT), 5-hydroxycotinine (5HC), cotinine-glucuronide (COT-glucuronide), 3-hydroxycotinine-glucuronide (3HC-glucuronide), cotinine-N-oxide (COT-N-oxide) (CNO), Norcotinine, Nornicotine, Nicotine-N-oxide (NNO), Nicotine-glucuronide, Anabaseine, Anatabine, Myosmine, Nicotelline, and Nicotyrine since the IC50 (>5,000 ng/mL) of binding of the antibody to each of these compounds is at least 12,500% greater than the IC50 (39.8 ng/mL) of binding of the antibody to 3-hydroxycotinine (Table 3).

The terms “antigen,” “immunogen,” “antigenic,” “immunogenic,” “antigenically active,” “immunologic,” and “immunologically active” when made in reference to a molecule, refer to any substance that is capable of inducing a specific humoral immune response (including eliciting a soluble antibody response) and/or cell-mediated immune response (including eliciting a CTL response). To elicit antibody production, in one embodiment, small molecules, or haptens, may be conjugated to keyhole limpet hemocyanin (KLH), bovine serum albumin (BSA) or fused to glutathione-S-transferase (GST).

The terms “affinity” and “functional affinity” when made in reference to the binding of two molecules (e.g. antibody (Ab) to an antigen (Ag), etc.) refer to an interaction of the two molecules that is dependent upon the presence of a particular structure on one or both of the molecules. Affinity is a measure of the overall strength of the chemical interaction between the antibody binding site and its monovalent antigen. It represents the balance between the ease with which an interaction occurs and the probability that the complex will dissociate and can be written as: Ab+Ag [symbol] AbAg. Written another way,

$${K}_{a} = \frac{{k}_{a}}{{k}_{d}} = \frac{[ AbAg ]}{[ Ab ] [ Ag ]}$$

where kₐ and k_{d} are the association and dissociation rate constants, respectively, and Kₐ is the equilibrium (affinity) constant. At equilibrium, when half of the antibody binding sites are occupied, Kₐ is equal to the reciprocal of the concentration of free antigen

$$( {K}_{a} = \frac{1}{[ Ag ]} ) .$$

Means of measuring or estimating affinity include immunoassay, equilibrium dialysis, and surface plasmon resonance. If the antibody-antigen interactions will take place in a competitive immunoassay, especially if it is a solid phase assay with immobilized antibody and a complex conjugate as label, affinity (as functional affinity) may be taken as being functionally equivalent to sensitivity and assessed by comparing the errors and slopes of standard curves.

For example, if an antibody has affinity or functional affinity for epitope “A” on the molecule, then the presence of a protein containing epitope A (or free, unlabelled A) in a reaction containing labeled “A” and the antibody will reduce the amount of labeled A bound to the antibody. Thus, in one embodiment, the terms “affinity” and “functional affinity” are relative terms referring to the ratio (e.g., percentage) of the level of binding of an antibody to a first molecule (e.g., 3-hydroxycotinine) relative to the level of binding of the antibody to a second molecule (e.g., nicotine and/or nicotine metabolite and/or tobacco alkaloid). Thus, as used herein, “affinity” of an antibody to a first molecule (e.g., 3-hydroxycotinine) refers to a level of binding of the antibody to a second molecule (e.g., nicotine and/or nicotine metabolite and/or tobacco alkaloid) that is 20% or less (including from 0% to 20%, from 0% to 19%, from 0% to 18%, from 0% to 17%, from 0% to 16%, from 0% to 15%, from 0% to 14%, from 0% to 13%, from 0% to 12%, from 0% to 11%, from 0% to 10%, from 0% to 9%, from 0% to 8%, from 0% to 7%, from 0% to 6%, from 0% to 5%, from 0% to 4%, from 0% to 3%, from 0% to 2%, and from 0% to 1% less) than the level of binding of the antibody to the first molecule. Said differently, “high affinity” or “high functional affinity” of an antibody to a first molecule (e.g., 3-hydroxycotinine) refers to a level of binding of the antibody to the first molecule that is at least five (5) fold greater (including from 5 to 1,000, from 5 to 500, from 5 to 100, from 5 to 50, from 5 to 25, and from 5 to 10 fold greater) than the level of binding of the antibody to a second molecule (e.g., nicotine and/or nicotine metabolite and/or tobacco alkaloid). In one embodiment, the level of binding of an antibody to a molecule is determined using the IC50 value. For example, the exemplary anti-3-hydroxycotinine antibody 22H9C7 has high functional affinity to 3-hydroxycotinine since the IC50 (>5,000 ng/mL) of binding of the antibody to nicotine, cotinine (COT), 5-hydroxycotinine (5HC), cotinine-glucuronide (COT-glucuronide), 3-hydroxycotinine-glucuronide (3HC-glucuronide), cotinine-N-oxide (COT-N-oxide) (CNO), Norcotinine, Nornicotine, Nicotine-N-oxide (NNO), Nicotine-glucuronide, Anabaseine, Anatabine, Myosmine, Nicotelline, and Nicotyrine is less than 1% of the IC50 (39.8 ng/mL) of binding of the antibody to 3-hydroxycotinine (Table 3).

A “cognate antigen” when used in reference to an antigen that binds to an antibody, refers to an antigen that is capable of specifically binding to the antibody.

In one embodiment, the antigen comprises an epitope. The terms “epitope” and “antigenic determinant” refer to a structure on an antigen, which interacts with the binding site of an antibody or T cell receptor as a result of molecular complementarity. An epitope may compete with the intact antigen, from which it is derived, for binding to an antibody.

As used herein the terms “portion” and “fragment” when made in reference to a nucleic acid sequence or protein sequence refer to a piece of that sequence that may range in size from 2 contiguous nucleotides and amino acids, respectively, to the entire sequence minus one nucleotide and amino acid, respectively.

A “subject” that may benefit from the invention's methods includes any multicellular animal, preferably a mammal. Mammalian subjects include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla. The invention's compositions and methods are also useful for a subject “in need of reducing one or more symptoms of” a disease includes a subject that exhibits and/or is at risk of exhibiting one or more symptoms of the disease. For Example, subjects may be at risk based on family history, genetic factors, environmental factors, etc. This term includes animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable). The invention's compositions and methods are also useful for a subject “at risk” for disease refers to a subject that is predisposed to contracting and/or expressing one or more symptoms of the disease. This predisposition may be genetic (e.g., a particular genetic tendency to expressing one or more symptoms of the disease, such as heritable disorders, etc.), or due to other factors (e.g., environmental conditions, exposures to detrimental compounds, including carcinogens, present in the environment, etc.). The term subject “at risk” includes subjects “suffering from disease,” i.e., a subject that is experiencing one or more symptoms of the disease. It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

“Sample” and “specimen” as used herein are used in their broadest sense to include any composition that is obtained and/or derived from a biological source, as well as sampling devices (e.g., swabs), which are brought into contact with biological or environmental samples. “Biological samples” include those obtained from a subject, including body fluids (such as urine, blood, plasma, fecal matter, cerebrospinal fluid (CSF), semen, sputum, and saliva), as well as solid tissue. Biological samples also include a cell (such as cell lines, cells isolated from tissue whether or not the isolated cells are cultured after isolation from tissue, fixed cells such as cells fixed for histological and/or immunohistochemical analysis), tissue (such as biopsy material), cell extract, tissue extract, and nucleic acid (e.g., DNA and RNA) isolated from a cell and/or tissue, and the like. These examples are illustrative, and are not to be construed as limiting the sample types applicable to the present invention.

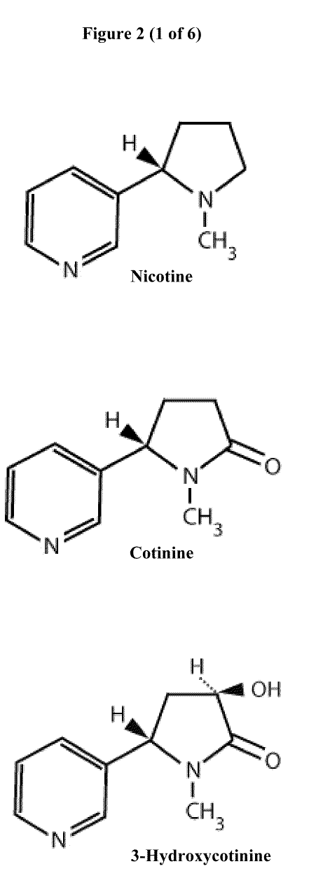

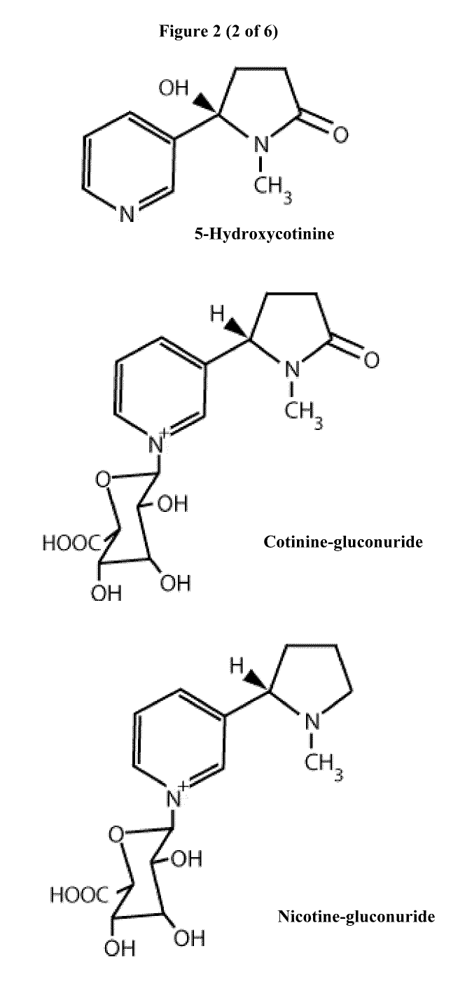

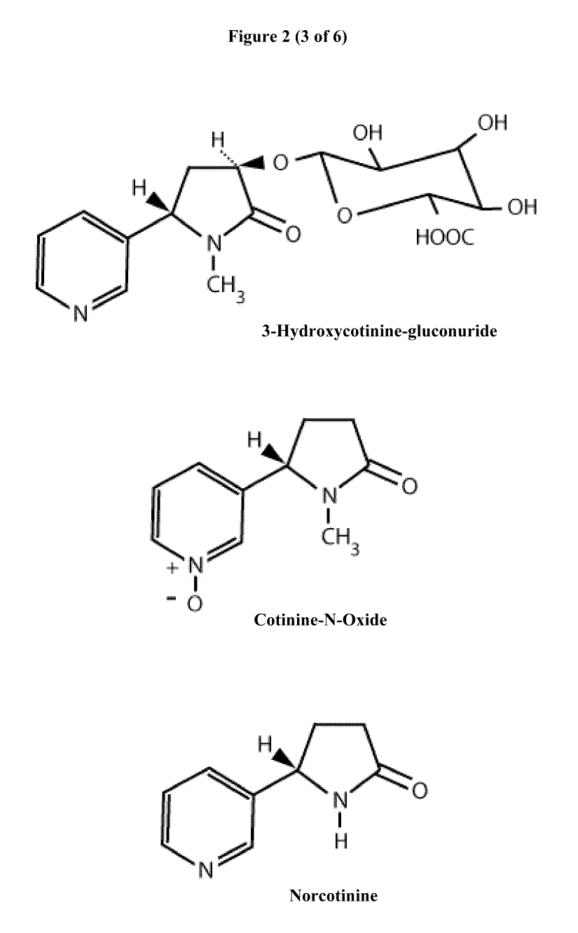

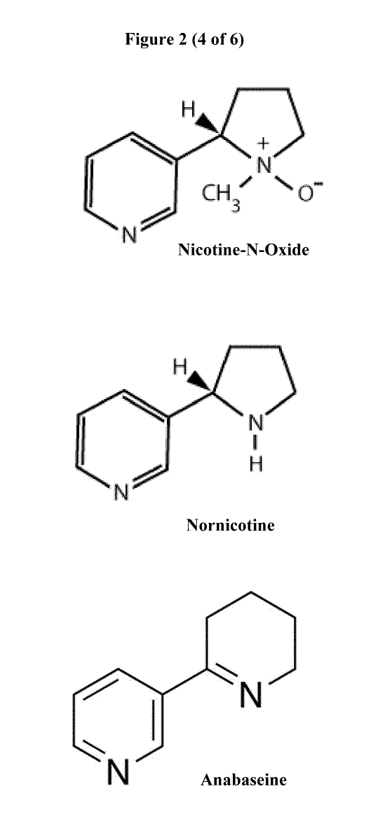

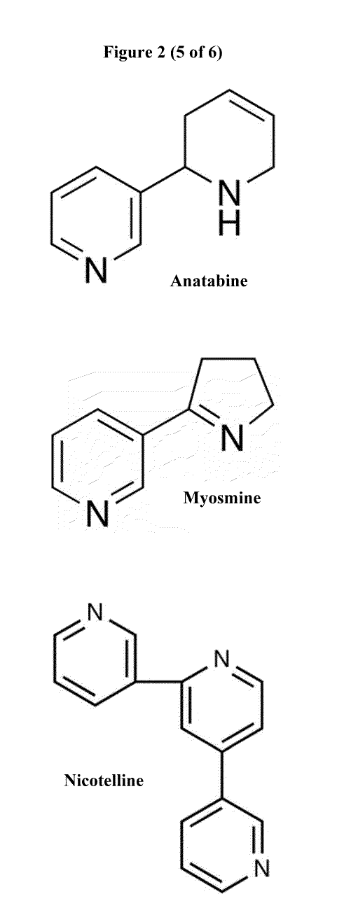

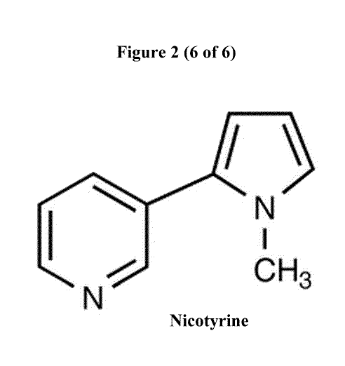

“Nicotine metabolite” refers to a product of metabolism of nicotine by a body tissue and/or body fluid. Nicotine metabolites include, but are not limited to cotinine (COT), 3-hydroxycotinine (3HC), 5-hydroxycotinine (5HC), cotinine-glucuronide (COT-glucuronide), 3-hydroxycotinine-glucuronide (3HC-glucuronide), cotinine-N-oxide (COT-N-oxide) (CNO), Norcotinine, Nornicotine, Nicotine-N-oxide (NNO), and Nicotine-glucuronide (FIG. 2).

“Cotinine” and “COT” are used interchangeably (FIG. 2).

“3-Hydroxycotinine” and “3HC” are used interchangeably (FIG. 2).

“5-Hydroxycotinine” and “5HC” are used interchangeably (FIG. 2).

“Tobacco alkaloid” refers to chemical compounds that exist naturally in tobacco. These compounds are transferred to smokers or smokeless tobacco users through contact with tobacco and are not usually products of metabolism, however, some tobacco alkaloids are also nicotine metabolites which occur both naturally in tobacco and are synthesized through metabolism of nicotine. Nicotine is an example of a tobacco alkaloid while Nornicotine is both a tobacco alkaloid and a nicotine metabolite. Tobacco alkaloids include, but are not limited to Nicotine, Nornicotine, Anabaseine, Anatabine, Nicotelline, and Nicotyrine (FIG. 2).

“Enzyme immunoassay” and “EIA” are interchangeably used to refer to a method for detecting and/or determining the level of an antibody or an antigen in a sample. The antibody or antigen is conjugated to an enzyme, and the antibody and antigen are contacted under conditions for binding of the antibody to the antigen. An enzymatic substrate is added that the enzyme can convert to a detectable signal (e.g., chromogenic, fluorogenic, electrochemiluminescent, etc.) The level of antibody, or antigen, that is linked to the enzyme is quantified by measuring the level of the detectable signal. Enzyme immunoassays are exemplified by, but not limited to, an “enzyme-linked immunosorbent assay” (“ELISA”). Performing an ELISA involves at least one antibody with specificity for a particular antigen. The sample with an unknown amount of antigen is immobilized on a solid support (usually a polystyrene microtiter plate) either non-specifically (via adsorption to the surface) or specifically (via capture by another antibody specific to the same antigen, in a “sandwich” ELISA). After the antigen is immobilized, the detection antibody is added, forming a complex with the antigen. The detection antibody can be covalently linked to an enzyme, or can itself be detected by a secondary antibody that is linked to an enzyme through bio-conjugation. Between each step the plate is typically washed with a mild detergent solution to remove any proteins or antibodies that are not specifically bound. After the final wash step the plate is developed by adding an enzymatic substrate to produce a detectable signal, which indicates the quantity of antigen in the sample.

The terms “reduce,” “inhibit,” “diminish,” “suppress,” “decrease,” and grammatical equivalents (including “lower,” “smaller,” etc.) when in reference to the level of any molecule (e.g., amino acid sequence, and nucleic acid sequence, antibody, etc.), cell, and/or phenomenon (e.g., disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of molecule, cell and/or phenomenon in the first sample (or in the first subject) is lower than in the second sample (or in the second subject) by any amount that is statistically significant using any art-accepted statistical method of analysis. In one embodiment, the quantity of molecule, cell and/or phenomenon in the first sample (or in the first subject) is at least 10% lower than, at least 25% lower than, at least 50% lower than, at least 75% lower than, and/or at least 90% lower than the quantity of the same molecule, cell and/or phenomenon in the second sample (or in the second subject). In another embodiment, the quantity of molecule, cell, and/or phenomenon in the first sample (or in the first subject) is lower by any numerical percentage from 5% to 100%, such as, but not limited to, from 10% to 100%, from 20% to 100%, from 30% to 100%, from 40% to 100%, from 50% to 100%, from 60% to 100%, from 70% to 100%, from 80% to 100%, and from 90% to 100% lower than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The terms “increase,” “elevate,” “raise,” and grammatical equivalents (including “higher,” “greater,” etc.) when in reference to the level of any molecule (e.g., amino acid sequence, and nucleic acid sequence, antibody, etc.), cell, and/or phenomenon (e.g., disease symptom, binding to a molecule, specificity of binding of two molecules, affinity of binding of two molecules, specificity to disease, sensitivity to disease, affinity of binding, enzyme activity, etc.) in a first sample (or in a first subject) relative to a second sample (or relative to a second subject), mean that the quantity of the molecule, cell and/or phenomenon in the first sample (or in the first subject) is higher than in the second sample (or in the second subject) by any amount that is statistically significant using any art-accepted statistical method of analysis. In one embodiment, the quantity of the molecule, cell and/or phenomenon in the first sample (or in the first subject) is at least 10% greater than, at least 25% greater than, at least 50% greater than, at least 75% greater than, and/or at least 90% greater than the quantity of the same molecule, cell and/or phenomenon in the second sample (or in the second subject). This includes, without limitation, a quantity of molecule, cell, and/or phenomenon in the first sample (or in the first subject) that is at least 10% greater than, at least 15% greater than, at least 20% greater than, at least 25% greater than, at least 30% greater than, at least 35% greater than, at least 40% greater than, at least 45% greater than, at least 50% greater than, at least 55% greater than, at least 60% greater than, at least 65% greater than, at least 70% greater than, at least 75% greater than, at least 80% greater than, at least 85% greater than, at least 90% greater than, and/or at least 95% greater than the quantity of the same molecule, cell and/or phenomenon in the second sample (or in the second subject). In one embodiment, the first subject is exemplified by, but not limited to, a subject that has been manipulated using the invention's compositions and/or methods. In a further embodiment, the second subject is exemplified by, but not limited to, a subject that has not been manipulated using the invention's compositions and/or methods. In an alternative embodiment, the second subject is exemplified by, but not limited to, a subject to that has been manipulated, using the invention's compositions and/or methods, at a different dosage and/or for a different duration and/or via a different route of administration compared to the first subject. In one embodiment, the first and second subjects may be the same individual, such as where the effect of different regimens (e.g., of dosages, duration, route of administration, etc.) of the invention's compositions and/or methods is sought to be determined in one individual. In another embodiment, the first and second subjects may be different individuals, such as when comparing the effect of the invention's compositions and/or methods on one individual participating in a clinical trial and another individual in a hospital.

The terms “alter” and “modify” when in reference to the level of any molecule and/or phenomenon refer to an increase or decrease.

Reference herein to any numerical range expressly includes each numerical value (including fractional numbers and whole numbers) encompassed by that range. To illustrate, and without limitation, reference herein to a range of “at least 50” includes whole numbers of 50, 51, 52, 53, 54, 55, 56, 57, 58, 59, 60, etc., and fractional numbers 50.1, 50.2 50.3, 50.4, 50.5, 50.6, 50.7, 50.8, 50.9, etc. In a further illustration, reference herein to a range of “less than 50” includes whole numbers 49, 48, 47, 46, 45, 44, 43, 42, 41, 40, etc., and fractional numbers 49.9, 49.8, 49.7, 49.6, 49.5, 49.4, 49.3, 49.2, 49.1, 49.0, etc. In yet another illustration, reference herein to a range of from “5 to 10” includes each whole number of 5, 6, 7, 8, 9, and 10, and each fractional number such as 5.1, 5.2, 5.3, 5.4, 5.5, 5.6, 5.7, 5.8, 5.9, etc.

### DESCRIPTION OF THE INVENTION

The invention provides antibodies, and antigen-binding fragments thereof, that specifically bind to 3-hydroxycotinine (3HC). The invention's antibodies and compositions containing them are useful in diagnostic and therapeutic applications for diseases involving nicotine activity (e.g., smoking and/or smokeless tobacco use). The invention is further described under A) Invention's antibodies, B) Cells, C) Kits, D) Diagnostic applications, and E) Therapeutic applications.

### A. Invention's Antibodies

The health risks of smoking have led to broad smoking cessation efforts in the workplace and other public settings. Nicotine is metabolized to cotinine, and cotinine further metabolized to 3-hydroxycotinine by the liver enzyme cytochrome P450 (CYP) 2A6. (Lerman 2006) It has been reported that the ratio of the two major nicotine metabolites, cotinine (COT) and 3-hydroxycotinine (3HC) provides the most reliable phenotypic measurement of CYP2A6 activity towards nicotine, and therefore, of nicotine metabolic rate. The 3HC/COT ratio has been shown to be an effective predictor of therapeutic response to transdermal nicotine. (Lerman 2006) For example, a faster nicotine metabolism rate, as indicated by a higher pre-treatment 3HC/COT ratio, may lead to lower nicotine blood levels from nicotine replacement products and poorer smoking cessation (SC) outcomes with standard-dose nicotine replacement therapy (2). Because the ratio of 3HC/COT is fairly constant over time (1), it may be useful in clinical practice for screening individual smokers in order to provide the most effective smoking cessation treatment for each person. Measurement of the 3HC/COT ratio currently is done by gas chromatography/mass spectrometry only. However, the process for GC/MS analysis is time-consuming, complex, expensive, and requires highly trained personnel. Thus, there remains a need for compositions and methods for more rapid, less expensive, easier to use, and more specific tests for the measurement of nicotine metabolic rate.

The invention addresses these needs by providing, in one embodiment, an antibody, or an antigen-binding fragment thereof, that specifically binds to 3-hydroxycotinine (3HC). The invention's antibodies may be used to determine the level of 3-hydroxycotinine in a sample, e.g. for determining the activity of the liver enzyme cytochrome P450 (CYP) 2A6 in metabolizing nicotine to cotinine and/or to 3-hydroxycotinine, and/or in metabolizing cotinine to 3-hydroxycotinine.

The invention's antibodies may also be used to determine the ratio of 3-hydroxycotinine to cotinine. This ratio may be used for phenotyping the activity of the liver enzyme cytochrome P450 (CYP) 2A6 and, thus, the rate of nicotine metabolism. In addition, the ratio of 3-hydroxycotinine to cotinine may be used as an aid for clinicians in dosing more effective and appropriate forms of nicotine replacement therapy (Lerman et al. (2006) Clin. Pharmacol. & Therapeutics 79:600-608; Mooney et al., (2008) Cancer Epidemiol. Biomarkers Prev., 17(6):1396-1400; Schnoll et al. (2009) Pharmacol Biochem Behav, 2009. 92(1): 6-11)

The invention's antibodies are also useful as affinity purification agents. In this process, the antibodies are immobilized on a suitable support, such a Sephadex resin or filter paper, using methods well known in the art, to capture and purify molecules that contain antigens that specifically bind to the invention's antibodies.

The invention's antibodies are also useful in therapeutic applications for treating disease associated with nicotine activity.

The invention's anti-3-hydroxycotine antibodies have specificity for the antigen 3-hydroxycotinine. “Specificity” refers to the proportion (e.g., percentage) of negatives that are correctly identified as such (e.g., the percentage of samples that are correctly identified as not containing a molecule, the percentage of healthy people who are correctly identified as not having a condition, etc.).

In one embodiment, specificity of an antibody for an antigen may be determined using IC50 of the antigen. “Half maximal inhibitory concentration” and “IC50” are interchangeably used to refer to the concentration of a substance (e.g., inhibitor, antagonist, etc.) that produces a 50% inhibition of a given biological process, or a component of a process (e.g., an enzyme, antibody, cell, cell receptor, microorganism, etc.). It is commonly used as a measure of an antagonist substance's potency. The IC50 of a substance can be determined by constructing a dose-response curve and examining the effect of different concentrations of the antagonist substance on reversing agonist activity. IC50 values can be calculated for a given antagonist substance by determining the concentration needed to inhibit half of the maximum biological response of the agonist. IC50 may be used to calculate % reactivity. In contrast, “half maximal effective concentration,” and “EC50” are interchangeably used to refer to the concentration of a substance (e.g., agonist, drug, antibody, toxicant, etc.) that produces 50% of a maximum biological process, or a component of a process (e.g., an enzyme, antibody, cell, cell receptor, microorganism, etc.). It is commonly used as a measure of a substance's potency. The EC50 of a substance can be determined by constructing a dose-response curve and examining the effect of different concentrations of the agonist substance on a biological process. EC50 values can be calculated for a given agonist substance by determining the concentration needed to produce half of the maximum biological process produced by the agonist substance.

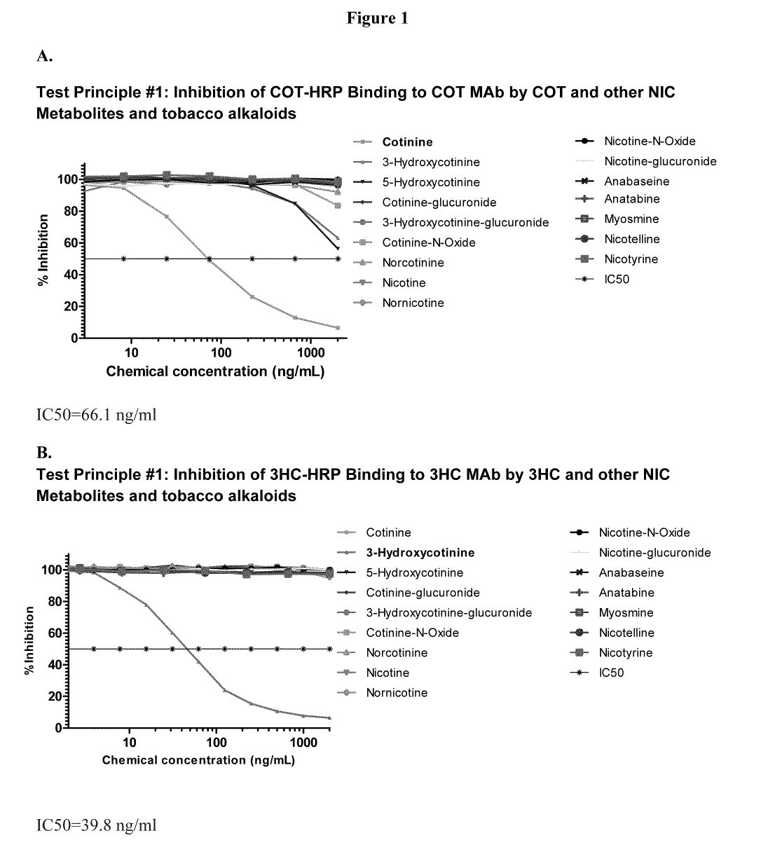

In one embodiment, the invention's anti-3-hydroxycotinine antibody, or the antigen-binding fragment thereof, has IC50 to 3-hydroxycotinine of from 10 to 500 ng/ml, including from 10 to 400, from 10 to 300, and/or from 10 to 200 ng/ml. For example, FIG. 1B shows that one of the invention's exemplary anti-3-hydroxycotinine antibodies has IC50 of 39.8 ng/ml for 3-hydroxycotinine.

Surprisingly, the invention's monoclonal antibodies were capable of detecting a 3-hydroxycotinine epitope that distinguished between the antigen 3-hydroxycotinine, and other nicotine metabolites such as cotinine and 5-hydroxycotinine. For example, in one embodiment, the invention's anti-3-hydroxycotinine antibody, or the antigen-binding fragment thereof, does not have substantial reactivity with (i.e., does not substantially bind) any of fifteen non-3HC NIC metabolites and tobacco alkaloids tested at concentrations from 1 to 2000 ng/ml, including from 1 to 1500, from 1 to 1000, and from 1 to 500 ng/ml. For example, the exemplary anti-3-hydroxycotinine monoclonal antibody 22H9C7 did not show binding to any of fifteen other NIC metabolites and tobacco alkaloids tested at concentrations from 1 ng/ml to 2000 ng/ml (FIG. 1B). As illustrated in Table 3, when % reactivity is calculated using the IC50 values, the exemplary anti-3-hydroxycotinine antibody 22H9C7 has 100% reactivity to 3-hydroxycotinine and less than 1% reactivity to the other fifteen NIC metabolites and tobacco alkaloids tested.

The exemplary anti-cotinine monoclonal antibody 3B11A4, has 100% reactivity to cotinine (IC50 66.1 ng/ml), but also has 1.86% reactivity to 3-hydroxycotinine (IC50 3545.0 ng/ml) and 4.39% reactivity to 5HC (IC50 1505.2 ng/ml) as shown in FIG. 1A and Table 2. The exemplary anti-cotinine monoclonal antibody 3B11A4 has less than 1.5% reactivity to the other thirteen NIC metabolites and tobacco alkaloids tested (Table 2).

The invention's anti-3-hydroxycotinine antibodies are sensitive to the antigen 3-hydroxycotinine. “Sensitivity” refers to the proportion (e.g., percentage) of actual positives that are correctly identified as such (e.g., the percentage of samples that are correctly identified as containing a molecule, the percentage of sick people who are correctly identified as having a condition, etc.). In one embodiment, sensitivity of an antibody to a molecule refers to the lowest concentration of the molecule that competes with, and reduces, binding of the antibody to its antigen. In one embodiment, the invention's anti-3-hydroxycotinine antibody, or the antigen-binding fragment thereof, has a sensitivity to 3-hydroxycotinine from 1 to 2000 ng/ml, including from 1 to 1000, from 1 to 500, from 1 to 100, from 1 to 50, from 1 to 20 and from 10 to 20. For example, data herein show an exemplary anti-3-hydroxycotinine antibody having a sensitivity to 3-hydroxycotinine of from 10 to 500 ng/ml (FIG. 1B).

The invention contemplates polyclonal antibodies, monoclonal antibodies, chimeric antibodies, recombinant antibodies, humanized antibodies, and antibodies displayed upon the surface of a phage (See U.S. Pat. No. 7,202,346). Generic methods for using phage display technology to produce anti-cotinine antibodies are disclosed in Park et al., U.S. Pat. Publ. No. US 2008/0226650. In one embodiment, the invention's antibodies are monoclonal antibodies produced by hybridoma cells. In a particular embodiment, the monoclonal antibody is exemplified by 22H9C7, 2C1006A4, 8D8E4, 14F12D3C8, 20C8B7, 22C8D6, 23A6F2, 23A7B2, and 25D8B4B2 (Table 1). In a particularly preferred embodiment, the monoclonal antibody comprises antibody 22H9C7 (Table 1, FIG. 1B).

In particular, the invention contemplates antibody fragments that contain the idiotype (“antigen-binding fragment”) of the antibody molecule. For example, such fragments include, but are not limited to, the Fab region, F(ab′)2 fragment, pFc′ fragment, and Fab′ fragments.

The “Fab region” and “fragment, antigen binding region,” interchangeably refer to portion of the antibody arms of the immunoglobulin “Y” that function in binding antigen. The Fab region is composed of one constant and one variable domain from each heavy and light chain of the antibody. Methods are known in the art for the construction of Fab expression libraries (Huse et al., Science, 246:1275-1281 (1989)) to allow rapid and easy identification of monoclonal Fab fragments with the desired specificity. In another embodiment, Fc and Fab fragments can be generated by using the enzyme papain to cleave an immunoglobulin monomer into two Fab fragments and an Fc fragment. The enzyme pepsin cleaves below the hinge region, so a “F(ab′)2 fragment” and a “pFc′ fragment” is formed. The F(ab′)2 fragment can be split into two “Fab′ fragments” by mild reduction.

The invention also contemplates a “single-chain antibody” fragment, i.e., an amino acid sequence having at least one of the variable or complementarity determining regions (CDRs) of the whole antibody, and lacking some or all of the constant domains of the antibody. These constant domains are not necessary for antigen binding, but constitute a major portion of the structure of whole antibodies. Single-chain antibody fragments are smaller than whole antibodies and may therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies. Techniques for the production of single-chain antibodies are known (U.S. Pat. No. 4,946,778). The variable regions of the heavy and light chains can be fused together to form a “single-chain variable fragment” (“scFv fragment”), which is only half the size of the Fab fragment, yet retains the original specificity of the parent immunoglobulin.

The “Fc” and “Fragment, crystallizable” region interchangeably refer to portion of the base of the immunoglobulin “Y” that function in role in modulating immune cell activity. The Fc region is composed of two heavy chains that contribute two or three constant domains depending on the class of the antibody. By binding to specific proteins, the Fc region ensures that each antibody generates an appropriate immune response for a given antigen. The Fc region also binds to various cell receptors, such as Fc receptors, and other immune molecules, such as complement proteins. By doing this, it mediates different physiological effects including opsonization, cell lysis, and degranulation of mast cells, basophils and eosinophils. In an experimental setting, Fc and Fab fragments can be generated in the laboratory by cleaving an immunoglobulin monomer with the enzyme papain into two Fab fragments and an Fc fragment.

The invention contemplates polyclonal antibodies and monoclonal antibodies. “Polyclonal antibody” refers to an immunoglobulin produced from more than a single clone of plasma cells (e.g., B-lymphocytes); in contrast “monoclonal antibody” (“MAb”) refers to an immunoglobulin produced from a single clone of plasma cells.

Generic methods are available for making polyclonal and monoclonal antibodies that are specific to a desirable polypeptide or small molecule. For the production of polyclonal antibodies, an antigen is injected into a mammal (such as a mouse, rabbit, goat, etc.). This induces the B-lymphocytes to produce IgG immunoglobulins specific for the antigen. This polyclonal IgG is purified from the mammal's serum.

For the production of monoclonal and polyclonal antibodies, various host animals can be immunized by injection with the peptide or small molecule corresponding to any molecule of interest in the present invention, including but not limited to rabbits, mice, rats, sheep, goats, etc. For preparation of monoclonal antibodies, any technique that provides for the production of antibody molecules by continuous cell lines in culture may be used (See e.g., Harlow and Lane, Antibodies: A Laboratory Manual, Cold Spring Harbor Laboratory Press, Cold Spring Harbor, N.Y.). These include, but are not limited to, the hybridoma technique originally developed by Köhler and Milstein (Köhler and Milstein, Nature, 256:495-497 (1975)), techniques using germ-free animals and utilizing technology such as that described in PCT/US90/02545, as well as the trioma technique, the human B-cell hybridoma technique (See e.g., Kozbor et al., Immunol. Today, 4:72 (1983)), and the EBV-hybridoma technique, to produce human monoclonal antibodies (Cole et al., in Monoclonal Antibodies and Cancer Therapy, Alan R. Liss, Inc., pp. 77-96 (1985)). In some particularly preferred embodiments of the present invention, the present invention provides monoclonal antibodies.

Also contemplated are chimeric antibodies. As used herein, the term “chimeric antibody” contains portions of two different antibodies, typically of two different species. See, e.g.: U.S. Pat. No. 4,816,567 to Cabilly et al.; U.S. Pat. No. 4,978,745 to Shoemaker et al.; U.S. Pat. No. 4,975,369 to Beavers et al.; and U.S. Pat. No. 4,816,397 to Boss et al. Chimeric antibodies include monovalent, divalent or polyvalent immunoglobulins. A monovalent chimeric antibody is a dimer (HL) formed by a chimeric H chain associated through disulfide bridges with a chimeric L chain. A divalent chimeric antibody is tetramer (H₂L₂) formed by two HL dimers associated through at least one disulfide bridge. A polyvalent chimeric antibody can also be produced, for example, by employing a Hc region that aggregates (e.g., IgM H chain).

The invention also contemplates “humanized antibodies,” i.e., chimeric antibodies that have constant regions derived substantially or exclusively from human antibody constant regions, and variable regions derived substantially or exclusively from the sequence of the variable region from a mammal other than a human. Humanized antibodies preferably have constant regions and variable regions other than the complement determining regions (CDRs) derived substantially or exclusively from the corresponding human antibody regions and CDRs derived substantially or exclusively from a mammal other than a human. Humanized antibodies may be generated using methods known in the art, including using human hybridomas (Cote et al. Proc. Natl. Acad. Sci. U.S.A.80:2026-2030 (1983)) or by transforming human B cells with EBV virus in vitro (Cole et al. in Monoclonal Antibodies and Cancer Therapy, Alan R. Liss, pp. 77-96 (1985)). Additional methods include, for example, generation of transgenic non-human animals which contain human immunoglobulin chain genes and which are capable of expressing these genes to produce a repertoire of antibodies of various isotypes encoded by the human immunoglobulin genes (U.S. Pat. Nos. 5,545,806; 5,569,825 and 5,625,126). Humanized antibodies may also be made by substituting the complementarity determining regions of, for example, a mouse antibody, into a human framework domain (PCT Pub. No. WO92/22653). Chimeric antibodies containing amino acid sequences that are fused to constant regions from human antibodies, or to toxins or to molecules with cytotoxic effect, are known in the art (e.g., U.S. Pat. Nos. 7,585,952; 7,227,002; 7,632,925; 7,501,123; 7,202,346; 6,333,410; 5,475,092; 5,585,499; 5,846,545; 7,202,346; 6,340,701; 6,372,738; 7,202,346; 5,846,545; 5,585,499; 5,475,092; 7,202,346; 7,662,387; 6,429,295; 7,666,425; and 5,057,313).

Antibodies that are specific for a particular antigen may be screened using methods known in the art (e.g., U.S. Pat. No. 7,202,346) and disclosed herein. For example, In the production of antibodies, screening for the desired antibody can be accomplished by radioimmunoassay, ELISA (enzyme-linked immunosorbent assay), “sandwich” immunoassays, immunoradiometric assays, gel diffusion precipitin reactions, immunodiffusion assays, in situ immunoassays (e.g., using colloidal gold, enzyme or radioisotope labels), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many means are known in the art for detecting binding in an immunoassay and are within the scope of the present invention. As is well known in the art, the immunogenic peptide should be provided free of the carrier molecule used in any immunization protocol. For example, if the peptide was conjugated to KLH, it may be conjugated to BSA, or used directly, in a screening assay.

In one embodiment, the invention's antibodies are monoclonal antibodies produced by a hybridoma cell line, as exemplified by the antibodies that recognized 3HC without cross reacting with COT-BSA and carrier protein KLH (e.g., 22H9C7, 2C1006A4, 8D8E4, 14F12D3C8, 20C8B7, 22C8D6, 23A6F2, 23A7B2, and 25D8B4B2 (Table 1). In a preferred embodiment, the antibody is 22H9C7 (Table 1, FIG. 1B).

In some applications, it may be desirable to conjugate the invention's anti-3-hydroxycotinine antibodies, or the antigen-binding fragment thereof, with a detectable label. “Probe” and “label” are interchangeably used to describe a chemical moiety that, when attached to a composition of interest, acts as a marker for the presence of the composition of interest, such that detection of the label corresponds to detection of the composition of interest.

The invention's compositions and methods are not limited to a particular approach to detecting binding of the invention's antibodies to their antigens. In one embodiment, detecting binding to the invention's antibodies typically involves using antibodies that are labeled with a radioisotope (e.g., ³H, ¹⁴C, ³²P, ³⁵S and/or ¹²⁵I), fluorescent or chemiluminescent compound (e.g., fluorescein isothiocyanate, rhodamine, and/or luciferin) and/or an enzyme (e.g., alkaline phosphatase, beta-galactosidase and/or horseradish peroxidase). Methods for conjugating antibodies to a detectable moiety are known in the art (e.g., Hunter, et al., Nature 144:945 (1962); David, e at., Biochemistry 13:1014 (1974); Pain, et al., J. Immunol. Meth. 40:219 (1981); and Nygren, J. Histochem. and Cytochem. 30:407 (1982).

Thus, the invention's antibodies may be employed in immunoassays, such as competitive binding assays, direct and indirect sandwich assays, and immunoprecipitation assays, including immunohistochemistry, enzyme-linked immunosorbent assay (ELISA), fluorescence-activated cell sorting (FACS), and Western blots.

### B. Cells

In addition to the invention's novel antibodies, the invention also provides cells that produce these antibodies. These are exemplified by B-lymphocytes for polyclonal antibody production, and by hybridoma cells for monoclonal antibody production. “Hybridoma cell” refers to a cell line produced by fusing a specific antibody-producing B cell with a myeloma (B cell cancer) cell that is selected for its ability to grow in tissue culture and for an absence of antibody chain synthesis. The antibodies produced by the hybridoma cell are all of a single specificity and are therefore monoclonal antibodies (in contrast to polyclonal antibodies).

### C. Kits

The invention also provides kits comprising i) any one or more of the anti-3-hydroxycotinine antibodies, or the antigen-binding fragment thereof, that are disclosed herein.

The term “kit” is used in reference to a combination of reagents and other materials. It is contemplated that the kit may include reagents such as buffering agents, nucleic acid stabilizing reagents, protein stabilizing reagents, signal producing systems (e.g., fluorescence generating systems such as fluorescence resonance energy transfer (FRET) systems, radioactive isotopes, etc.), antibodies, control antibodies, antigens, as well as testing containers (e.g., microtiter plates, etc.). It is not intended that the term “kit” be limited to a particular combination of reagents and/or other materials. In one embodiment, the kit further comprises instructions for using the reagents. The test kit may be packaged in any suitable manner, typically with the elements in a single container or various containers as necessary along with a sheet of instructions for carrying out the test. In some embodiments, the kits also preferably include a positive control sample. Kits may be produced in a variety of ways that are standard in the art. In some embodiments, the kits contain at least one reagent for detecting and/or quantifying the level of an antigen. In preferred embodiments, the instructions specify that the recommended type and dose of smoking cessation therapeutic may be determined by determining the ratio of 3-hydroxycotine to cotinine in a sample from the subject.

In one embodiment, the kit further comprises ii) an anti-cotinine antibody, or an antigen-binding fragment thereof, that specifically binds to cotinine. In another embodiment, the kit further comprises iii) instructions for using the anti-3-hydroxycotinine antibody, or using the antigen-binding fragment thereof, for determining the level of 3-hydroxycotinine in a sample. In yet another embodiment, the kit further comprises instructions for using the anti-cotinine antibody, or using an antigen-binding fragment thereof, for determining the level of cotinine in a sample. In a further embodiment, the kit further comprises instructions for determining the ratio of 3HC/COT in a sample. In yet another embodiment, the kit further comprises recommendations for therapy, such as smoking cessation (SC) aids and/or nicotine replacement therapy (NRT) dosing based on the ratio of 3HC/COT in the sample.

### D. Diagnostic Applications

The invention's anti-3-hydroxycotinine antibodies may be used in methods for detecting 3-hydroxycotinine in a sample, comprising a) providing i) any one or more of the anti-3-hydroxycotinine antibodies, or the antigen-binding fragment thereof, that are disclosed herein, ii) a test sample, b) contacting the sample with the anti-3-hydroxycotinine antibody, or with the antigen-binding fragment thereof under conditions for specific binding of the anti-3-hydroxycotinine antibody, or binding of the antigen-binding fragment thereof, with 3-hydroxycotinine, and c) detecting binding of the anti-3-hydroxycotinine antibody, or binding of the antigen-binding fragment thereof, to the sample. These methods may be used to determine the level of 3-hydroxycotinine in a sample, e.g. for determining the activity of the liver enzyme cytochrome P450 (CYP) 2A6 in metabolizing nicotine to cotinine and/or to 3-hydroxycotinine, and/or in metabolizing cotinine to 3-hydroxycotinine.

Thus, in one embodiment, the method further comprises d) determining the level of 3-hydroxycotinine that binds to the anti-3-hydroxycotinine antibody, or binds to the antigen-binding fragment thereof, thereby determining the level of 3-hydroxycotinine in the sample.

To facilitate detection, the anti-3-hydroxycotinine antibody, or the antigen-binding fragment thereof, may comprise a detectable label, such as a label that is detectable in an enzyme-linked immunoassay (ELISA) (Langley et al., U.S. Pat. No. 7,553,630), or in a radioimmunoassay.

In particular embodiments, the methods of the invention are used to determine the ratio of 3-hydroxycotinine to cotinine in a sample. In such applications, the methods further comprise e) providing an anti-cotinine antibody, or an antigen-binding fragment thereof, that specifically binds to cotinine, f) contacting the sample with the anti-cotinine antibody, or with the antigen-binding fragment thereof, under conditions for specific binding of the anti-cotinine antibody with cotinine, and g) determining the level of cotinine that binds to the anti-cotinine antibody or binds to the antigen-binding fragment thereof. In another embodiment, the method further comprises h) determining the ratio of the level of the 3-hydroxycotinine from step d) and the level of the cotinine from step g). Methods for making anti-cotinine antibodies are known in the art (Park et al., U.S. Pat. Publ. No. US 2008/0226650).

The ratio of 3-hydroxycotinine to cotinine that is determined in accordance with the invention's methods may be used for phenotyping the activity of the liver enzyme cytochrome P450 (CYP) 2A6 and, thus, the rate of nicotine metabolism. In addition, the ratio of 3-hydroxycotinine to cotinine may be used as an aid for determining the most effective form and/or dose of smoking cessation therapy (Lerman et al. (2006) Clin. Pharmacol. & Therapeutics 79:600-608; Mooney et al., (2008) Cancer Epidemiol. Biomarkers Prev., 17(6):1396-1400; Schnoll et al. (2009) Pharmacol Biochem Behav, 2009. 92(1): 6-11.

The 3-hydroxycotinine/cotinine ratio is useful as a biomarker to predict success with nicotine treatment (e.g., nicotine patch) for smoking cessation (Schnoll et al. (2009)). For example, Controlling for sex, race, age, and nicotine dependence, smokers in the upper 3 quartiles of 3-hydroxycotinine/cotinine ratio (faster metabolizers) were approximately 50% less likely to be abstinent vs. smokers in the first quartile (slow metabolizers; 28% vs. 42%; OR=0.54 (95% CI: 0.36-0.82), p=0.003). Among abstainers, plasma nicotine levels (assessed 1 week after treatment began) decreased linearly across the 3-hydroxycotinine/cotinine ratio (β=−3.38, t(355)=−3.09, pb.05) (Schnoll et al. (2009)).

In one embodiment, the 3-hydroxycotinine/cotinine (“3HC/COT”) ratio may be obtained using a competitive ELISA designed for the quantitative measurement of 3-hydroxycotinine and cotinine levels in biological samples, such as human plasma, serum, saliva, and urine. The concentrations of cotinine and 3-hydroxycotinine in unknown samples will be calculated from standard curves developed with each analyte, and ratios of 3HC/COT are therefore obtained. In one embodiment suitable for clinical use, the assay may be developed in a 96-well microplate format, using one or more of the following test principle #1, test principle #2, and test principle #3.
In test principle #1, the anti-3-hydroxycotinine antibody is attached to a solid substrate, and detecting binding of the anti-3-hydroxycotinine antibody, or binding of the antigen-binding fragment thereof, to the sample comprises contacting the sample with a 3-hydroxycotine that comprises a detectable label. (Webb et al. U.S. Pat. No. 7,521,198).

Thus in one embodiment, a solid support (e.g., plate wells) are coated separately with the invention's anti-3-hydroxycotinine antibody and with anti-cotinine antibody. Various concentrations of standards (authentic cotinine and 3-hydroxycotinine) and test samples containing unknown amounts of cotinine and/or 3-hydroxycotinine are incubated with the immobilized antibodies in the presence of a constant amount of labeled 3-hydroxycotinine (e.g., 3-hydroxycotinine-horseradish peroxidase) and labeled cotinine (e.g., cotinine-horseradish peroxidase), respectively. After incubation, components that did not bind to the antibodies are washed away. The amount of labeled 3-hydroxycotinine (e.g., 3-hydroxycotinine-horseradish peroxidase) and labeled cotinine (e.g., cotinine-horseradish peroxidase) that bound to the immobilized antibodies be detected and measured by the reaction of the horseradish peroxidase label with a peroxidase substrate. Optical density (OD) at 450 nm is read with a multiwell spectrophotometer and two standard curves are generated using the OD values from authentic 3-hydroxycotinine and cotinine of known concentrations. Levels of 3-hydroxycotinine and cotinine in the test samples are calculated from the standard curves, followed by generation of a ratio of 3-hydroxycotinine to cotinine. This method is exemplified in Examples 2, 3, 4 and 5. Example 4 also shows an exemplary standardization of the assay using known amounts of cotinine and 3-hydroxycotinine mixed in serum. Example five illustrates the generation of the 3HC/COT ratio using results if the enzyme immunoassay by test principle #1 using known amounts of cotinine and 3-hydroxycotinine mixed in serum.

In test principle #2, the anti-3-hydroxycotinine antibody is in aqueous solution, and detecting binding of the anti-3-hydroxycotinine antibody, or binding of the antigen-binding fragment thereof, to the sample comprises contacting the sample with a second antibody that specifically binds to the anti-3-hydroxycotinine antibody, or specifically binds to the antigen-binding fragment thereof, wherein the second antibody comprises a detectable label (Webb et al., U.S. Pat. No. 7,521,198).

Thus, in one embodiment, a solid support (e.g., plate wells) is individually coated with 3-hydroxycotinine-BSA and cotinine-KLH. The invention's anti-3-hydroxycotinine antibody and anti-cotinine antibody at an optimal concentration are pre-incubated with known amounts of standards (authentic cotinine and 3-hydroxycotinine) and with test samples containing unknown amounts of cotinine and/or 3-hydroxycotinine. Mixtures are then contacted with the immobilized 3-hydroxycotinine-BSA and cotinine-KLH (e.g., immobilized on plate wells) for antibody-antigen binding. After incubation, components that did not bind to the antibodies are washed away. The amount of the invention's anti-3-hydroxycotinine antibody and anti-cotinine antibody that bound to the immobilized 3-hydroxycotinine (e.g., 3HC-BSA) and to the immobilized cotinine (e.g., COT-KLH) may be measured with anti-mouse IgG-horseradish peroxidase (IgG-HRP). Apparent 3-hydroxycotinine and cotinine concentrations are inversely related to the OD450 value. Levels of 3-hydroxycotinine and cotinine in the test samples are calculated from the standard curves, followed by generation of a ratio of 3-hydroxycotinine to cotinine. In test principle #3, regardless of whether the antibodies are immobilized (as in principle #1) or in aqueous solution (as in principle #2), the detectable label is conjugated to the antibody instead of (or in addition) to the antigen. Thus, in test principle #3, at least one of the anti-3-hydroxycotinine antibody, the antigen-binding fragment of the anti-3-hydroxycotinine antibody, the anti-cotinine antibody, and the antigen-binding fragment of the anti-cotinine antibody, comprises a detectable label, and wherein binding of the anti-3-hydroxycotinine antibody, or binding of the antigen-binding fragment thereof, to the sample comprises detecting the detectable label (Webb et al., U.S. Pat. No. 7,521,198).

The invention also provides a database comprising a ratio of the level of 3-hydroxycotinine and the level of cotinine determined using any of methods described herein. The database of the ratio of 3-hydroxycotinine and cotinine may be used to recommend the most effective form and/or dose of smoking cessation therapy (Lerman et al. (2006) Clin. Pharmacol. & Therapeutics 79:600-608; Mooney et al., (2008) Cancer Epidemiol. Biomarkers Prev., 17(6):1396-1400; Schnoll et al. (2009) Pharmacol Biochem Behav, 2009. 92(1): 6-11).

### E. Therapeutic Applications

The invention provides methods for treating disease associated with nicotine activity comprising administering to a subject in need thereof, and/or having the disease, a therapeutically effective amount of any of the invention's anti-3-hydroxycotinine antibodies. In one embodiment, the method further comprises detecting a reduced level of 3-hydroxycotinine in a sample from the subject after administering the invention's compositions, compared to control samples, e.g., prior to administering the invention's compositions. In a further embodiment, the method may further comprise detecting a reduction in one or more symptom of the disease following administering the invention's compositions.

Generic methods for treating disease with antibodies are known in the art (Park et al., U.S. Pat. Publ. No. US 2008/0226650 for treating disease associated with nicotine activity using anti-nicotine and anti-cotinine antibodies; Payne et al., U.S. Pat. No. 7,202,346 for treating cancer with anti-MUC16 antibodies; U.S. Pat. Nos. 6,333,410; 5,475,092; 5,585,499; 5,846,545; 7,202,346; 6,340,701 & 6,372,738; 7,662,387; 7,662,387; 7,662,387; 6,429,295; 7,666,425; 5,057,313). In particular, antibody treatment of human beings with cancer is known in the art, for example in U.S. Pat. Nos. 5,736,137; 6,333,410; 5,475,092; 5,585,499; 5,846,545; 7,202,346; 6,340,701; 6,372,738; 7,202,346; 5,846,545; 5,585,499; 5,475,092; 7,202,346; 7,662,387; 7,662,387; 6,429,295; 7,666,425; 5,057,313.

The term “administering” to a subject means providing a molecule to a subject. This may be done using methods known in the art (e.g., Erickson et al., U.S. Pat. No. 6,632,979; Furuta et al., U.S. Pat. No. 6,905,839; Jackobsen et al., U.S. Pat. No. 6,238,878; Simon et al., U.S. Pat. No. 5,851,789). The invention's compositions may be administered prophylactically (i.e., before the observation of disease symptoms) and/or therapeutically (i.e., after the observation of disease symptoms). Administration also may be concomitant with (i.e., at the same time as, or during) manifestation of one or more disease symptoms. Also, the invention's compositions may be administered before, concomitantly with, and/or after administration of another type of drug or therapeutic procedure (e.g., nicotine patch). Methods of administering the invention's compositions include, without limitation, administration in parenteral, oral, intraperitoneal, intranasal, topical and sublingual forms. Parenteral routes of administration include, for example, subcutaneous, intravenous, intramuscular, intrasternal injection, and infusion routes.

In one embodiment, the invention's compositions comprise a lipid for delivery as liposomes. Methods for generating such compositions are known in the art (Borghouts et al. (2005). J Pept Sci 11, 713-726; Chang et al. (2009) PLoS One 4, e4171; Faisal et al. (2009) Vaccine 27, 6537-6545; Huwyleret al. (2008) Int J Nanomedicine 3, 21-29; Song et al. (2008) Int J Pharm 363, 155-161; Voinea et al. J Cell Mol Med 6, 465-474).

The invention's antibodies may be administered with pharmaceutically acceptable carriers, diluents, and/or excipients. Examples of suitable carriers, diluents and/or excipients include: (1) Dulbecco's phosphate buffered saline, pH about 7.4, containing about 1 mg/ml to 25 mg/ml human serum albumin, (2) 0.9% saline (0.9% w/v NaCl), and (3) 5% (w/v) dextrose.

The invention's antibodies are typically administered in a therapeutic amount. The terms “therapeutic amount,” “pharmaceutically effective amount,” “therapeutically effective amount,” and “biologically effective amount,” are used interchangeably herein to refer to an amount that is sufficient to achieve a desired result, whether quantitative or qualitative. In particular, a pharmaceutically effective amount is that amount that results in the reduction, delay, and/or elimination of undesirable effects (such as pathological, clinical, biochemical and the like) that are associated with disease.

For example, specific “dosages” of a “therapeutic amount” will depend on the route of administration, the type of subject being treated, and the physical characteristics of the specific subject under consideration. These factors and their relationship to determining this amount are well known to skilled practitioners in the medical, veterinary, and other related arts. This amount and the method of administration can be tailored to achieve optimal efficacy but will depend on such factors as weight, diet, concurrent medication and other factors, which those skilled in the art will recognize. The dosage amount and frequency are selected to create an effective level of the compound without substantially harmful effects.

When present in an aqueous dosage form, rather than being lyophilized, the antibody typically will be formulated at a concentration of about 0.1 mg/ml to 100 mg/ml.

Depending on the type and severity of the disease, about 0.015 to 15 mg of antibody/kg of patient weight is an initial candidate dosage for administration to the patient, whether, for example, by one or more separate administrations, or by continuous infusion. For repeated administrations over several days or longer, depending on the condition, the treatment is repeated until a desired suppression of disease symptoms occurs.

“Treating” a disease refers to reducing one or more symptoms (such as objective, subjective, pathological, clinical, sub-clinical, etc.) of the disease.

A “subject” that may benefit from the invention's methods includes any multicellular animal, preferably a mammal. Mammalian subjects include humans, non-human primates, murines, ovines, bovines, ruminants, lagomorphs, porcines, caprines, equines, canines, felines, ayes, etc.). Thus, mammalian subjects are exemplified by mouse, rat, guinea pig, hamster, ferret and chinchilla. The invention's compositions and methods are also useful for a subject “in need of reducing one or more symptoms of” a disease, including animal models of the disease. Thus, administering a composition (which reduces a disease and/or which reduces one or more symptoms of a disease) to a subject in need of reducing the disease and/or of reducing one or more symptoms of the disease includes prophylactic administration of the composition (i.e., before the disease and/or one or more symptoms of the disease are detectable) and/or therapeutic administration of the composition (i.e., after the disease and/or one or more symptoms of the disease are detectable). It is not intended that the present invention be limited to any particular signs or symptoms. Thus, it is intended that the present invention encompass subjects that are experiencing any range of disease, from sub-clinical symptoms to full-blown disease, wherein the subject exhibits at least one of the indicia (e.g., signs and symptoms) associated with the disease.

### EXPERIMENTAL

The following examples serve to illustrate certain preferred embodiments and aspects of the present invention and are not to be construed as limiting the scope thereof.

### Example 1

### Screening and Selection of Anti-3-Hydroxycotinine Monoclonal Antibodies

To generate 3-hydroxycotinine monoclonal antibodies, we chose rac-trans-3-hydroxycotinine-3-carboxylic acid as a precursor for the antigen preparation. This 3HC derivative was custom synthesized by Toronto Research Chemicals Inc. and conjugated to a carrier protein, keyhole limpet hemocyanin (KLH). The conjugate (3HC-KLH) was used as an immunogen to raise hybridomas for 3-hydroxycotinine monoclonal antibody production.

Test bleeds were checked for the quality and strength of the immune response in an indirect ELISA and the result showed the immune sera bound to 3HC-BSA with minimum cross-reactivity to COT-BSA. Hybridoma clones specific for 3HC were subsequently generated using hybridoma technology.

In particular, hybridomas were screened against 3HC-KLH, COT-BSA, and KLH concurrently by an indirect ELISA. MAbs that recognized 3HC without cross reacting with the COT-BSA and carrier protein KLH were selected (e.g., 22H9C7, 2C1006A4, 8D8E4, 14F12D3C8, 20C8B7, 22C8D6, 23A6F2, 23A7B2, and 25D8B4B2. Some of the monoclonal antibodies are listed in Table 1.

TABLE 1
3-hydroxycotinine monoclonal antibody
Screening by Indirect ELISA.*

| | 3HC-BSA | COT-BSA | KLH |
|---|---|---|---|
| Clone Name | (OD 450 nm against immobilized targets) | | |
| 2C10 C6 A4 | 3.355 | 0.068 | 0.012 |
| 8D8 E4 | 3.033 | 0.01 | 0.019 |
| 14F12 D3C8 | 3.331 | 0.107 | −0.022 |
| 20C8 B7 | 3.094 | 0.071 | 0.057 |
| 22C8 D6 | 3.174 | −0.011 | −0.001 |
| 22H9 C7 | 2.939 | 0.008 | 0.018 |
| 23A6 F2 | 2.655 | 0.01 | 0.049 |
| 23A7 B6 | 3.169 | 0.038 | 0.004 |
| 25D8 B4B2 | 3.881 | 0.294 | 0.029 |

*96-well plates were coated with different antigens at 5 ug/ml. Hybridoma supernatants were applied and incubated with the immobilized antigens. Anti mouse IgG-HRP was used for detection.

### Example 2_{[MH1]}

### Validation of Sensitivity and Specificity of Anti-Cotinine Monoclonal Antibodies by Test Principle #1

Specificity and sensitivity of the anti-cotinine monoclonal antibody (COT MAb) were determined in competitive microplate ELISA with COT and a battery of NIC metabolites and tobacco alkaloids using COT-HRP as tracer. The nicotine metabolites and/or tobacco alkaloids include COT, 3HC, 5HC, COT-glucuronide, 3HC-glucuronide, COT-N-oxide, Norcotinine, Nornicotine, Nicotine-N-oxide, Nicotine-glucuronide, Anabaseine, Anatabine, Myosmine, Nicotelline, and Nicotyrine. FIG. 1A illustrates an inhibition curve using the anti-cotinine monoclonal antibody (3B11A4) that was formed with various concentrations of authentic COT. Apparent COT concentrations are inversely related to the OD450 value. Specificity is illustrated by the very low level of reactivity to other nicotine metabolites and tobacco alkaloids exhibited by the anti-cotinine monoclonal antibody (Table 2). IC50 values are shown in FIG. 1A and Table 2.

TABLE 2
Reactivity of the Cotinine MAb 3B11A4 with nicotine,
nicotine metabolites, and tobacco alkaloids.*

| Competitor | IC50 (ng/mL) | Reactivity (%) |
|---|---|---|
| Cotinine | 66.1 | 100 |
| 3-Hydroxycotinine | 3545 | 1.86 |
| 5-Hydroxycotinine | 1505.2 | 4.39 |
| Cotinine-gluconuride | >5,000 | <1.5 |
| 3-Hydroxycotinine-gluconuride | >5,000 | <1.5 |
| Cotinine-N-Oxide | >5,000 | <1.5 |
| Norcotinine | >5,000 | <1.5 |
| Nicotine | >5,000 | <1.5 |
| Nornicotine | >5,000 | <1.5 |
| Nicotine-N-Oxide | >5,000 | <1.5 |
| Nicotine-gluconuride | >5,000 | <1.5 |
| Anabaseine | >5,000 | <1.5 |
| Anatabine | >5,000 | <1.5 |
| Myosmine | >5,000 | <1.5 |
| Nicotelline | >5,000 | <1.5 |
| Nicotyrine | >5,000 | <1.5 |

$* \% Reactivity = \frac{COT IC50}{metabolite IC50} \times 100$

FIG. 1A and Table 2 show that % reactivity of the anti-cotinine monoclonal antibody (3B11A4) for COT was 100%, while % reactivity was only 1.86% for 3HC and 4.39% for 5HC. The anti-cotinine monoclonal antibody showed less than 1.5% reactivity for the thirteen other NIC metabolites and tobacco alkaloids.

### Example 3

### Validation of Sensitivity and Specificity of Anti-3-Hydroxycotinine Monoclonal Antibodies by Test Principle #1

As for the anti-cotinine monoclonal antibody, above, specificity and sensitivity of the anti-3-hydroxycotinine monoclonal antibody (3HC MAb) was determined in competitive microplate ELISA with 3HC and a battery of NIC metabolites and tobacco alkaloids using 3HC-HRP as tracer. The nicotine metabolites and/or tobacco alkaloids include COT, 3HC, 5HC, COT-glucuronide, 3HC-glucuronide, COT-N-oxide, Norcotinine, Nornicotine, Nicotine-N-oxide, Nicotine-glucuronide, Anabaseine, Anatabine, Myosmine, Nicotelline, and Nicotyrine. FIG. 1B illustrates an inhibition curve using the anti-3-Hydroxycotinine monoclonal antibody (22H9C7) that was formed with various concentrations of authentic 3HC. Apparent 3HC concentrations are inversely related to the OD450 value.

TABLE 3
Reactivity of the 3-Hydroxycotinine MAb 22H9C7 with nicotine,
nicotine metabolites, and tobacco alkaloids.*

| Competitor | IC₅₀ (ng/mL) | Reactivity (%) |
|---|---|---|
| Cotinine | >5,000 | <1 |
| 3-Hydroxycotinine | 39.8 | 100 |
| 5-Hydroxycotinine | >5,000 | <1 |
| Cotinine-gluconuride | >5,000 | <1 |
| 3-Hydroxycotinine-gluconuride | >5,000 | <1 |
| Cotinine-N-Oxide | >5,000 | <1 |
| Norcotinine | >5,000 | <1 |
| Nicotine | >5,000 | <1 |
| Nornicotine | >5,000 | <1 |
| Nicotine-N-Oxide | >5,000 | <1 |
| Nicotine-gluconuride | >5,000 | <1 |
| Anabaseine | >5,000 | <1 |
| Anatabine | >5,000 | <1 |
| Myosmine | >5,000 | <1 |
| Nicotelline | >5,000 | <1 |
| Nicotyrine | >5,000 | <1 |

$* \% Reactivity = \frac{3 HC IC50}{metabolite IC50} \times 100$

FIG. 1B depicts the data obtained in the competition assay using one of the 3-hydroxycotinine monoclonal antibodies (22H9C7). Table 3 shows that % reactivity of the anti-3-hydroxycotinine monoclonal antibody (22H9C7) for 3HC was 100%, while % reactivity was less than 1% for the fourteen other NIC metabolites and tobacco alkaloids.

### Example 4

### Standardization of Enzyme Immunoassay for Determination of the Amounts of Cotinine and 3HC in Serum Using Test Principle #1

Wells were coated with an anti-cotinine monoclonal antibody and a 3-hydroxycotinine monoclonal antibody individually. Various concentrations of standards (authentic COT and 3HC) were incubated with the immobilized MAbs in the presence of a constant amount of antigen that is labeled with horseradish peroxidase (HRP), i.e., COT-HRP and 3HC-HRP, respectively. After incubation, unbound components were washed away. COT-HRP and 3HC-HRP that bound to the immobilized MAbs in the well were measured by the reaction of the HRP with peroxidase substrate. Optical density (OD) at 450 nm was read with a multiwell spectrophotometer. The results are shown in Table 4.

TABLE 4
Enzyme Immunoassay for determination of
the amounts of COT and 3HC in serum

| | Spiked | Observed | |
|---|---|---|---|
| Test | Concentration | Concentration | |
| Condition | (ng/mL) | (ng/mL) | % Recovery |
| Human serum | 15 | 19.619 | 130.8 |
| spiked | 35 | 33.077 | 94.5 |
| with COT | 80 | 86.706 | 108.4 |
| | 125 | 120.451 | 96.4 |
| | 225 | 229.752 | 102.1 |
| | 400 | 410.552 | 102.6 |
| | 500 | 531.177 | 106.2 |
| Human serum | 18 | 23.755 | 132 |
| spiked | 35 | 41.585 | 118.8 |
| with 3HC | 80 | 87.61 | 109.5 |
| | 125 | 135.504 | 108.4 |
| | 225 | 275.146 | 122.3 |
| | 400 | 411.037 | 102.8 |

The results in Table 4 show the accurate determination of the concentration of each of cotinine and 3HC in serum using the invention's antibodies and methods.

### Example 5

### Determination of the 3HC/Cot Ratio in Spiked Normal Serum Using Test Principle #1

Wells were coated with an anti-cotinine monoclonal antibody and a 3-hydroxycotinine monoclonal antibody individually. Various concentrations of standards (authentic COT and 3HC) and samples of known concentrations of COT and 3HC were incubated with the immobilized MAbs in the presence of a constant amount of antigen that is labeled with horseradish peroxidase (HRP), i.e., COT-HRP and 3HC-HRP, respectively. After incubation, unbound components were washed away. COT-HRP and 3HC-HRP that bound to the immobilized MAbs in the well were measured by the reaction of the HRP with peroxidase substrate. Optical density (OD) at 450 nm was read with a multiwell spectrophotometer. Concentrations of COT and 3HC in the samples were determined by comparison to the standard curves and the 3HC/COT ratio was calculated based on the observed concentrations. The results are shown in Table 5.

TABLE 5
3HC/COT Ratio Determination - Recovery of samples spiked into normal human serum.*

| Spiked | Spiked | Expected | Avg. Observed | Avg. 3HC/COT | Inter- |
|---|---|---|---|---|---|
| 3HC | COT | 3HC/COT | 3HC/COT Ratio | Ratio % | assay CV |
| (ng/mL) | (ng/mL) | Ratio | (SD) | Difference* | (%) |
| 5 | 20 | 0.25 | 0.233 (0.021) | 6.8 (lower) | 9.02 |
| 10 | 30 | 0.333 | 0.335 (0.047) | 0.6 (lower) | 13.88 |
| 15 | 35 | 0.429 | 0.451 (0.059) | 5.1 (lower) | 13.09 |
| 25 | 50 | 0.5 | 0.513 (0.046) | 2.6 (lower) | 9.06 |
| 35 | 60 | 0.583 | 0.544 (0.079) | 6.6 (lower) | 14.46 |
| 50 | 72 | 0.694 | 0.680 (0.054) | 2.0 (lower) | 8 |
| 75 | 94 | 0.798 | 0.809 (0.034) | 1.3 (higher) | 4.15 |
| 95 | 110 | 0.864 | 0.974 (0.190) | 12.7 (higher) | 19.54 |

$* Avg .3 HC / COT Ratio \% Difference = \frac{\begin{matrix}Expected 3 HC / COT Ratio - \\ Avg . Observed 3 HC / COT Ratio\end{matrix}}{Expected 3 HC / COT Ratio}$

### REFERENCES

1. Dempsey, D., et al., Nicotine metabolite ratio as an index of cytochrome P450 2A6 metabolic activity. Clin Pharmacol Ther, 2004. 76(1): p. 64.
2. Schnoll, R. A., et al., Nicotine metabolic rate predicts successful smoking cessation with transdermal nicotine: a validation study. Pharmacol Biochem Behav, 2009. 92(1): p. 6-11.
3. Rees, W. A., et al., Environmental Toxicology and Risk Assessment. Development and Characterization of an ELISA for trans-3-Hydroxycontinine, a Biomarker for Mainstream and Sidestream Smoke Exposure. Vol. Fifth. 1996.149-162.
4. U.S. Pat. No. 5,164,504, Walling, et al., “Haptens, tracers, immunogens and antibodies for immunoassays for cotinine derivatives. Nov. 17, 1992.

Each and every publication and patent mentioned in the above specification is herein incorporated by reference in its entirety for all purposes. Various modifications and variations of the described methods and system of the invention will be apparent to those skilled in the art without departing from the scope and spirit of the invention. Although the invention has been described in connection with specific embodiments, the invention as claimed should not be unduly limited to such specific embodiments. Indeed, various modifications of the described modes for carrying out the invention which are obvious to those skilled in the art and in fields related thereto are intended to be within the scope of the following claims.

We claim:

1. An isolated antibody, or an antigen-binding fragment thereof, that specifically binds to 3-hydroxycotinine (3-HC).

2. The antibody, or antigen-binding fragment thereof, of claim 1, wherein said antibody is selected from the group consisting of monoclonal antibody, chimeric antibody, recombinant antibody, humanized antibody, and an antibody displayed upon the surface of a phage.

3. The antibody, or antigen-binding fragment thereof, of claim 1, wherein said antibody is a monoclonal antibody produced by a hybridoma cell.

4. The antibody, or antigen-binding fragment thereof, of claim 1, wherein said antibody, or said antigen-binding fragment thereof, does not substantially bind to a molecule selected from the group consisting of nicotine nicotine metabolite, and tobacco alkaloid.

5. The antigen-binding fragment of claim 1, wherein said antigen-binding fragment is selected from the group consisting of a Fab fragment, a F(ab′)2 fragment, and a Fv fragment.

6. The antibody, or antigen-binding fragment thereof, of claim 1, wherein said anti-3-hydroxycotinine antibody, or said antigen-binding fragment thereof, comprises a label.

7. An isolated monoclonal antibody, or an antigen-binding fragment thereof, produced by a hybridoma cell, wherein said antibody specifically binds to 3-hydroxycotinine (3-HC).

8. A cell producing the antibody, or antigen-binding fragment thereof, of claim 1.

9. The cell of claim 8, wherein said cell is a hybridoma cell.

10. The cell of claim 8, wherein said cell is a B-lymphocyte.

11. A kit comprising the anti-3-hydroxycotinine antibody, or the antigen-binding fragment thereof, of claim 1.

12. The kit of claim 11, further comprising an anti-cotinine antibody, or an antigen-binding fragment thereof, that specifically binds to cotinine.

13. A method for detecting 3-hydroxycotinine in a sample, comprising
a) providing
i) the anti-3-hydroxycotinine antibody, or the antigen-binding fragment thereof, of claim 1, and
ii) a test sample,
b) contacting said sample with said anti-3-hydroxycotinine antibody, or with said antigen-binding fragment thereof, and
c) detecting binding of said anti-3-hydroxycotinine antibody, or binding of said antigen-binding fragment thereof, to said sample.

14. The method of claim 13, further comprising d) determining the level of 3-hydroxycotinine that binds to said anti-3-hydroxycotinine antibody, or binds to said antigen-binding fragment thereof.

15. The method of claim 13, wherein said anti-3-hydroxycotinine antibody, or said antigen-binding fragment thereof, comprises a label.

16. The method of claim 13, wherein said detecting comprises an enzyme immunoassay.

17. The method of claim 14, further comprising
e) providing an anti-cotinine antibody, or an antigen-binding fragment thereof, that specifically binds to cotinine,
f) contacting said sample with said anti-cotinine antibody, or with said antigen-binding fragment thereof, and
g) determining the level of cotinine that binds to said anti-cotinine antibody or binds to said antigen-binding fragment thereof.

18. The method of claim 17, further comprising
h) determining the ratio of the level of said 3-hydroxycotinine from step d) and the level of said cotinine from step g).

19. The method of claim 13, wherein said anti-3-hydroxycotinine antibody is attached to a solid substrate.

20. The method of claim 13, wherein said detecting comprises contacting said sample with a 3-hydroxycotine that comprises a label.

21. The method of claim 13, wherein said anti-3-hydroxycotinine antibody is in aqueous solution.

22. The method of claim 13, wherein said detecting comprises contacting said sample with a second antibody that specifically binds to said anti-3-hydroxycotinine antibody, or specifically binds to said antigen-binding fragment thereof, wherein said second antibody comprises a label.

23. The method of claim 17, wherein at least one of said anti-3-hydroxycotinine antibody, said antigen-binding fragment of said anti-3-hydroxycotinine antibody, said anti-cotinine antibody, and said antigen-binding fragment of said anti-cotinine antibody, comprises a label, and wherein said detecting comprises detecting said label.

24. A method for treating a disease associated with nicotine activity comprising administering to a subject in need thereof a therapeutically effective amount of the antibody, or antigen-binding fragment thereof, of claim 1.

25. The method of claim 24, further comprising detecting a reduction in the level of 3-hydroxycotinine in a sample from said subject after said administering compared to prior to said administering.

26. The method of claim 24, further comprising detecting a reduction in one or more symptom of said disease following said administering compared to prior to said administering.
